(19) United States
(12) Patent Application Publication
(10) Pub. No.: US 2024/0425412 A1
(43) Pub. Date: Dec. 26, 2024

(54) COMPOSITIONS AND METHODS UTILIZING ALTERNATIVE SOURCES OF CARBON DIOXIDE FOR SEQUESTRATION

(71) Applicant: CARBONCURE TECHNOLOGIES INC., Dartmouth (CA)

(72) Inventors: Dean Paul FORGERON, Bayside (CA); Casey James LEIST, Atlanta, GA (US); Robert NIVEN, Victoria (CA); George Sean MONKMAN, Halifax (CA)

(21) Appl. No.: 18/701,010

(22) PCT Filed: Oct. 12, 2022
(86) PCT No.: PCT/US2022/046459

§ 371 Date: Apr. 12, 2024

Related U.S. Application Data
(60) Provisional application No. 63/320,729, filed on Mar. 17, 2022.
(60) Provisional application No. 63/254,633, filed on Oct. 12, 2021.

(51) Int. Cl. C04B 20/02; B01D 53/62; C04B 18/167; C04B 40/02; C04B 111/00; C12P 5/02

(52) U.S. Cl. CPC C04B 20/023; B01D 53/62; C04B 18/167; C04B 40/0231; C12P 5/023; B01D 2251/404; B01D 2257/504; B01D 2258/05; B01D 2258/06; C04B 2111/00017

(57) ABSTRACT

Provided herein are systems and methods for use of carbon dioxide, such as carbon dioxide containing one or more contaminants, e.g., produced in processes where the carbon dioxide comes directly or indirectly from the atmosphere, in one or more systems to perform one or more processes that binds the carbon dioxide or one or more carbon dioxide reaction products with one or more reactants to produce one or more carbon dioxide sequestration products, for example, products that permanently sequester the carbon dioxide.

### CROSS-REFERENCE

This application claims the benefit of U.S. Provisional Application No. 63/254,633, filed Oct. 12, 2021, and U.S. Provisional Application No. 63/320,729 filed Mar. 17, 2022, which application is incorporated herein by reference.

### BACKGROUND

Sequestration and removal of carbon dioxide is becoming of increasing importance: new and more efficient methods of carbon dioxide removal and/or sequestration are needed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Carbon dioxide can be produced in various processes in which the carbon dioxide contains one or more contaminants, e.g., one or more contaminants whose nature and/or concentration render the carbon dioxide unfit for various uses, e.g., carbon dioxide that is not food grade, or not of sufficient purity for other uses. Among these processes are biogas production, syngas production, ethanol production, e.g., from landfill waste, biomass, hydrogen production, and the like. Generally the carbon dioxide associated with these processes is considered a waste or as needing further purification to be of use. In certain cases, the carbon dioxide is used without any additional purification. In certain cases, the carbon dioxide is further purified prior to use. In many of these processes, and others, the carbon dioxide is carbon dioxide that has been removed, directly or indirectly from the atmosphere. As used herein, the term “carbon dioxide removed directly or indirectly from the atmosphere,” and similar terms, includes carbon dioxide that is directly removed from the current atmosphere, e.g., by direct air capture (DAC) or carbon dioxide that has been indirectly removed, e.g., in a process, such as the production of biomass, where carbon dioxide from the current atmosphere is captured in, e.g., carbohydrates of the biomass, then, in a later process, such as a biogas process, an ethanol production process, a hydrogen production from biomass process, released from the biomass as new molecules of carbon dioxide: sequestration of such carbon dioxide is hoped to ultimately result in reduction of atmospheric carbon dioxide, or at least decrease the rate of increase of atmospheric carbon dioxide due to other process. Carbon dioxide directly or indirectly derived from the atmosphere does not include carbon dioxide produced from burning of fossil fuels, calcination of limestone to produce cement, or the like, as such carbon dioxide, even if it were indirectly produced from an atmosphere, the atmosphere was not the current atmosphere. The use of this carbon dioxide, including the one or more contaminants, in a process to sequester the carbon dioxide, preferably long-term, presents an opportunity to both ensure carbon removal and use of the carbon dioxide that, at present, is often considered unfit for use.

In addition, the facilities in which these processes are carried out, which can be referred to herein as a “carbon dioxide-producing facility,” and the like, are located in various geographic locations, often in proximity to other facilities where carbon dioxide from the CO2-producing processes can be utilized in carbon dioxide-sequestering processes. Such a facility can be referred to herein as a “sequestration plant.” “sequestration facility,” and the like. In general, in a sequestration facility, carbon dioxide, or a reaction product of carbon dioxide, is chemically reacted with one or more reactants to bind the carbon dioxide or reaction product, non-covalently or covalently, in a reaction product. Thus, a sequestration facility does not include, e.g., a facility for pumping carbon dioxide underground, e.g., for sequestration, enhanced oil recovery, and the like. In addition, even if such carbon dioxide reacts with minerals, e.g., underground, to create a reaction product, it is understood that the underground minerals are not part of the facility, thus, not a sequestration facility, as that term is used herein. If one or more carbon dioxide-producing facilities is networked with one or more carbon dioxide-sequestering facilities, a ready supply of carbon dioxide, requiring minimal or no treatment, and minimal transport, can be achieved. In some cases, the sequestration facility or facilities may utilize some or all of the carbon dioxide to produce a useful and/or commercially valuable product. In some cases, a sequestration facility can be a concrete production plant and/or a concrete product treatment plant, which, collectively, can be referred to herein as a concrete product plant: in general the term refers to a facility where one or more concrete products are produced and/or where one or more concrete products are treated, as described more fully herein. Thus, carbon dioxide, e.g., carbon dioxide comprising contaminants above a certain level, which is now considered a waste product or, at best, a product in need of extensive further purification for use, can be a feedstock as is, or with minimal purification, at low or no cost, and with minimal transportation costs and associated carbon dioxide production from the transportation.

A system can be established that includes: 1) one or carbon dioxide-producing facilities, e.g., one or more biogas facilities, one or more ethanol facilities, one or more direct air capture facilities, and/or one or more hydrogen production facilities, and the like, that are fitted with the necessary equipment to separate and/or treat separated carbon dioxide to render it suitable for transport, typically as is or with minimal purification (i.e., with one or more contaminants remaining in the carbon dioxide): 2) transportation equipment, suited to the form and volume of carbon dioxide, used to transport the separated and treated carbon dioxide; and 3) one or more carbon sequestration facilities to which the carbon dioxide is transported and where the carbon dioxide, usually without further purification or with minimal purification, is reacted with one or more materials that sequester the carbon dioxide and that, in many cases, also sequester one or more of the contaminants in the carbon dioxide. It will be appreciated that what is considered a “contaminant” of carbon dioxide for regulatory purposes may be relatively innocuous, or toxic, or anything in between: a relatively innocuous contaminant can be carried along with the separated carbon dioxide and even if not sequestered in subsequent reactions, its release is harmless, so long as it does not substantially interfere with the sequestration process. In some cases, more toxic contaminants, as described further herein, may be carried along and sequestered along with carbon dioxide, by chemical reaction, physical trapping, or a combination thereof. In certain embodiments, a single entity partially or wholly owns or controls the equipment at one or more of the carbon dioxide-producing facilities to separate the carbon dioxide and/or to render it transportable, in some cases, owns or controls the equipment that renders the carbon dioxide transportable: the same entity may partially or wholly own equipment at the carbon sequestration facilities for sequestering the carbon dioxide and, in some cases, one or more contaminants of the carbon dioxide. In certain embodiments, the carbon sequestration facilities can include one or more of 1) one or more systems to deliver carbon dioxide (and, in some cases, one or more contaminants in the carbon dioxide) to a mixing cement mix, e.g., concrete at a concrete production facility, in general, the mixing cement mix, e.g. concrete mix, is a mix produced from initial addition of water to dry cement powder: 2) one or more systems to deliver carbon dioxide (and, in some cases, one or more contaminants in the carbon dioxide) to wet concrete that has been, e.g., returned to a concrete production facility, or other wet concrete; in general, wet concrete is concrete that is still wet and capable of being mixed but is not a concrete mix during or immediately after initial addition of water to dry cement powder: 2) one or more systems to deliver carbon dioxide (and, in some cases, one or more contaminants in the carbon dioxide) to wash water from concrete production, transport, and/or use (also known as process water, or grey water) to carbonate the wash water: in some cases also one or more systems to deliver at least a portion of the carbonated wash water for use as mix water in a subsequent concrete batch: 3) one or more systems to deliver carbon dioxide (and, in some cases, one or more contaminants in the carbon dioxide) to recycled concrete aggregate and/or concrete aggregate fines. The carbon dioxide is converted to carbonate form, e.g., calcium carbonate, and permanently sequestered. One or more contaminants may be released, removed, or also sequestered with the carbon dioxide. As used herein, the term “permanently sequestered” refers to carbon dioxide that will not be released to the atmosphere in appreciable quantity during the normal use or storage of a product in which it is sequestered for hundreds or even thousands of years: it will be appreciated that carbon dioxide that is sequestered, e.g., as calcium carbonate in a solid mass such as a concrete block, is comparable to calcium carbonate in, e.g., limestone, and just as stable. This is merely exemplary. Carbon dioxide that can be released in a shorter time span during the normal use or storage of a product, e.g., in a matter of days, weeks, months, or a few years, can be referred to as “temporarily sequestered.” Additionally, or alternatively, the system further comprises one or more purification systems for purifying the carbon dioxide prior to rendering it suitable for transport, processing, and/or use. This can be advantageous when the desired equipment for use with the carbon dioxide requires that the carbon dioxide be of a certain purity, for example equipment requiring the carbon dioxide to be of food and beverage grade for use with the equipment. The purification system can be configured to produce any suitable purity of carbon dioxide as dictated by the requirements of the equipment, such as at least 50, 60, 70, 80, 90, 95, 99, 99.9, 99.99, 99.999, or 100% pure. In certain embodiments, the system comprises a first purification system comprising an inlet for a gas source comprising carbon dioxide, for example the waste gas from the biogas operation, a purified gas outlet for directing a gas stream comprising purified carbon dioxide, and a waste gas outlet for directing the waste gas depleted in carbon dioxide to a downstream operation and/or to atmosphere. In certain embodiments, the waste gas depleted in carbon dioxide has a higher concentration of one or more components of interest, for example a biogas component, e.g., methane, that can be either routed to a downstream operation, for example a biogas upgrader, or sent to a second purification system. In certain embodiments, the waste gas outlet is sent to a second purification system configured to increase the concentration of one or more components of interest, for example hydrogen or methane, that can be recovered for one or more commercial applications of interest.

It will be appreciated that, in the case of carbon dioxide sources where the carbon dioxide is obtained, directly or indirectly, from the atmosphere, such systems and processes will result in carbon removal from the atmosphere. In any case, the amount of carbon dioxide from, e.g., each source of carbon dioxide, along with carbon dioxide produced from various aspects of the systems and processes (e.g., carbon dioxide emitted during transport, carbon dioxide emitted during energy generation to power various processes, etc.), may be tracked and/or calculated, and a net carbon removal may be determined, for which carbon credits may be issued. The carbon credit may be shared between the owner or owners or controller or controllers of the carbon dioxide producing facilities and the owner or owners or controller or controller of the carbon sequestration systems, in any suitable manner.

Sources of carbon dioxide suitable for use in methods and compositions disclosed herein may be any suitable source. In certain embodiments, a source of carbon dioxide comprises a process or facility practicing a process in which carbon dioxide is produced that is, at least in part, directly or indirectly, from the atmosphere. An example of the former is direct air capture (DAC). Examples of the latter include biogas production: ethanol production, e.g., from plant feedstock, such as corn; and some forms of hydrogen production, e.g., hydrogen production from biomass. The carbon dioxide produced in these processes is often considered a byproduct of the main product, e.g., methane, in the case of biogas production, ethanol, in the case of ethanol production, and hydrogen, in the case of hydrogen production. Generally the carbon dioxide is released or otherwise goes to waste, in the normal course of the process. Other suitable sources of carbon dioxide, as known in the art, may also be used.

In certain embodiments, a source of carbon dioxide comprises a biogas facility. “Biogas,” as that term is used herein, includes a product of organic material decomposing in an anaerobic (zero oxygen) condition. The raw biogas generally needs treatment to clean out impurities to meet pipeline specifications. “Biomethane,” as that term is used herein, includes cleaned and conditioned biogas that meets pipeline quality. “Renewable Natural Gas (RNG),” as that term is used herein, includes pipeline compatible gaseous fuel derived from biogenic or other renewable sources that has lower life cycle carbon dioxide equivalent (CO2-eq) emissions than geological natural gas, often with the Environmental Attributes attached from a renewable source. As a substitute it can be used to replace natural gas in thermal applications, to generate electricity, for vehicle fuel or as a bio-product feedstock. “Environmental Attribute,” as that term is used herein, includes a volume of gas that was produced in a designated environmentally beneficial fashion. The “EAs” can be separated from the biomethane and assigned to gas in a different region (e.g., biomethane can be produced in Wisconsin, put in a pipeline, and receive credits for California's LCFS program)

A “biogas facility.” “biogas plant,” and other similar terms as used herein, includes a facility where a gas comprising methane and carbon dioxide is generated by anaerobic digestion of organic matter. A feedstock source or sources may be any suitable source, such as landfill, animal manure, materials produced at a water resource recovery facility, food waste, agriculture residue, forestry and forest product residue, agricultural plant wasted, municipal solid waste, and the like.

At the biogas facility, and/or at another location, methane is separated from carbon dioxide: the methane is generally separated and/or treated in such a way that it can be used as fuel, e.g., to be used in gas pipeline injection or other applications, e.g., as RNG. Carbon dioxide separated from the methane is often vented or otherwise discarded, in large part because it often contains one or more contaminants present at levels high enough that the carbon dioxide does not meet standards for industry or food use. Compositions and methods herein utilize carbon dioxide from biogas facilities: in certain cases a method herein comprises separating carbon dioxide from a biogas, in certain cases the carbon dioxide has already been separated and is provided. In either case, the carbon dioxide can be used as is, or subject to further purification, as appropriate. The carbon dioxide can be processed as necessary to render it suitable for transport. In some cases, the carbon dioxide may be liquified, and transported as a liquid. In some cases, the carbon dioxide may be gaseous, and transported as a gas, e.g., a pressurized gas. In some cases, some or all of the carbon dioxide, such as carbon dioxide treated to render it suitable for transport, may be stored at or near the biogas facility. A system may include a system to separate the carbon dioxide from methane, a system to process carbon dioxide to render it suitable for transport, one or more storage containers, and/or, in some cases, a system to purify the carbon dioxide, e.g., partially purify it, before rendering it suitable for transport. The purification system can be configured to produce any suitable purity of carbon dioxide, such as at least 50, 60, 70, 80, 90, 95, 99, 99.9, 99.99, 99.999, or 100% pure. In certain embodiments, included is a system, typically located at a biogas facility, to render carbon dioxide suitable for transport. The system may be supplied by a party other than the party that owns or controls the biogas facility.

Generally, the carbon dioxide will be transported to a site other than the biogas facility, though it is also possible that some or all of the carbon dioxide is used at the biogas facility, e.g., used to produce materials to sequester the carbon dioxide, e.g., permanently sequester the carbon dioxide, as described more fully elsewhere herein. If transported, any suitable method of transport may be used, such as via truck, pipeline, rail, barge, and/or any other suitable conveyance or combination of conveyances. In certain cases, the equipment used for transporting the carbon dioxide can require the carbon dioxide to be of a suitable purity, and, thus, the methods and systems described herein are configured to produce the requisite purity of carbon dioxide for use with the equipment, for example food and beverage grade CO2.

A biogas facility may be any suitable biogas facility. Types of biogas facilities include a landfill gas (LFG) biogas plant, animal manure biogas plant, water resource recovery facilities (WRRF) biogas plant, food waste biogas plant, agricultural residue biogas plant, forestry and/or forest product residue biogas plant, energy crops biogas plant, municipal solid waste (MSW) biogas plant, renewable electricity biogas plant, or a combination thereof. In certain embodiments a biogas facility comprises a LFG biogas plant. In certain embodiments a biogas facility comprises an animal manure biogas plant. In certain embodiments, a plurality of biogas facilities are used in a system or method provided herein, such as at least 2, 3, 4, 5, 6, 7, 8, 9, 10, 12, 15, 17, 20, 25, 30, 40, 50, 60, or 75 biogas facilities, and/or not more than 3, 4, 5, 6, 7, 8, 9, 10, 12, 15, 17, 20, 25, 30, 40, 50, 60, 75, or 100 biogas facilities.

The carbon dioxide, whether from a biogas facility or other facility as described further herein, typically contains contaminants, often of a type and/or at a concentration that the carbon dioxide is unsuitable for typical commercial use, e.g., unsuitable as a food grade carbon dioxide. In the United States, the FDA currently requires that a carbon dioxide be at least 99.90% pure for use in food. In certain embodiments, carbon dioxide used in compositions and methods disclosed herein is less than 99.99, 99.95, 99.92, 99.90, 98.99, 98.98, 98.97, 98.96, 98.95, 98.92, 98.90, 98.80, 98.70, 98.60, 98.5, 98.0, 97.5, 97.0, 96.5, 96.0, 95.5, 95.0, 94.5, 94.0, 93.5, 93.0, 92.5, 92.0, 91.5, 91.0, 90.5, 90.0, 89, 88, 87, 86, 85, 84, 83, 82, 81, 80, 77, 75, 72, 70, 65, 60, 55, or 55% pure. In certain embodiments, carbon dioxide is less than 99.90% pure. In certain embodiments, carbon dioxide is less than 99% pure. In certain embodiments, carbon dioxide is less than 95% pure. In certain embodiments, carbon dioxide is less than 90% pure. In certain embodiments, carbon dioxide is 80-99.89%, 80-95%, 80-90%, 85-99.89%, 85-95%, 85-90%, 90-99.89%, 90-95%, or 95-99.89% pure. In certain embodiments one or more contaminants are present in carbon dioxide at a total concentration of at least 0.001, 0.01, 0.09, 0.1, 0.2, 0.3, 0.4, 0.49, 0.5, 0.6, 0.7, 0.8, 0.9, 0.99, 1.0, 1.1, 1.2, 1.5, 1.7, 2, 2.5, 3, 3.5, 4, 4.5, 5, 6, 7, 8, 9, 10, 12, 15, or 20% (for example, % w/w), such as at least 0.09%, at least 0.99% contaminants or at least 2% contaminants.

The nature of contaminants and their individual concentrations in the carbon dioxide generally depends on its source. In carbon dioxide from a biogas facility, for example, carbon dioxide contaminants often depend at least in part on the on the type of organic matter used for anaerobic digestion. Thus, if animal or other manure is used, fecal matter contamination may be present. LFG may contain a variety of contaminants, depending on the contents of the landfill, etc. For example, the disposal of cosmetics and deodorants within household waste leads to siloxanes in the biogas, the presence of refrigerants leads to halogenated hydrocarbons in the biogas, and the operation of the landfill gas collection system under vacuum in an effort to limit fugitive emissions 60 often incidentally leads to the presence of nitrogen and oxygen in the biogas. Contaminants can include one or more of water vapor, nitrogen, oxygen, hydrogen, carbon monoxide, ammonia, sulfur compounds (in particular hydrogen sulfide), halogenated hydrocarbons, siloxanes, other volatile and semi-volatile organic compounds, heavy metals.

Another source of carbon dioxide is carbon dioxide associated with ethanol production, especially ethanol production from biomass, such as corn. Currently, carbon dioxide produced in the process, while relatively high in purity, still requires further purification before being used, e.g., as a food grade carbon dioxide. The systems and methods herein can utilize carbon dioxide produced in ethanol production as is, e.g., without any purification. It need merely be processed to render it suitable for transportation: if transported as a gas, this may be no more than pressurizing the carbon dioxide. In certain embodiments, a system or method herein utilizes carbon dioxide produced at an ethanol production facility, such as a corn ethanol production facility. A system or systems may be implemented at the facility to, e.g., render carbon dioxide transportable, store carbon dioxide, or the like. In certain embodiments, a system or method provided herein includes one or more ethanol production facilities and/or one or more systems within an ethanol production facility, such as a system to render carbon dioxide produced at the ethanol production facility transportable, and/or a system to store carbon dioxide. Transportation can be as described elsewhere herein. Additionally, or alternatively, the system further comprises one or more purification systems for purifying the carbon dioxide prior to rendering it suitable for transport, processing, and/or use. This can be advantageous when the desired equipment for use with the carbon dioxide requires that the carbon dioxide be of a certain purity, for example equipment requiring the carbon dioxide to be of food and beverage grade for use with the equipment. The purification system can be configured to produce any suitable purity of carbon dioxide as dictated by the requirements of the equipment, such as at least 50, 60, 70, 80, 90, 95, 99, 99.9, 99.99, 99.999, or 100% pure.

The carbon dioxide is transported, as necessary, to one or more sites where at least a portion of the carbon dioxide is sequestered, e.g., by chemically reacting it or a reaction product of it to form a carbon dioxide sequestration products or products. The product or products may be non-permanent sequestration products, such as paints, fuels, and the like. In certain embodiments, the product or products may be permanent sequestration products, such as mineralized products. Permanent sequestration of carbon dioxide can include conversion of carbon dioxide into thermodynamically stable products. In mineralization, carbon dioxide can be converted into a thermodynamically stable solid.

In certain cases, the carbon dioxide or a portion thereof is converted into thermodynamically stable products that are, or are part of, commercially valuable products that can be used in one or more industries.

In certain embodiments, the carbon dioxide, or a portion thereof, is used in one or more processes or systems associated with a concrete product. A site where one or more concrete products is produced or treated can be referred to herein as a “concrete product site.”

In certain embodiments, carbon dioxide from one or more carbon dioxide producers, such as carbon dioxide containing one or more contaminants as described herein, or a portion thereof, is used to carbonate a mixing concrete mix, generally during its initial mixing, e.g., during and/or shortly after water addition to dry cement. In these embodiments, carbon dioxide may be delivered to the mixing concrete mix in any suitable form, for example, as a mixture of solid and gaseous carbon dioxide which may, e.g., be produced from liquid carbon dioxide. Any suitable dosage of carbon dioxide may be used, e.g., 0.001-10% by weight cement (bwc), 0.001-5%, 0.001-3%, 0.001-2%, 0.001-1.5%, 0.001-1.0%, 0.001-0.8%, 0.001-0.5%, 0.001-0.1%, 0.005-10%, 0.005-5%, 0.005-3%, 0.005-2%, 0.005-1.5%, 0.005-1.0%, 0.005-0.8%, 0.005-0.5%, 0.005-0.1%, 0.01-10%, 0.01-5%, 0.01-3%, 0.01-2%, 0.01-1.5%, 0.01-1.0%, 0.01-0.8%, 0.01-0.5%, 0.01-0.1%, 0.1-10%, 0.1-5%, 0.1-3%, 0.1-2%, 0.1-1.5%, 0.1-1.4%, 0.1-1.3%, 0.1-1.2%, 0.1-1.0%, 0.1-0.9%, 0.1-0.8%, 0.1-0.7%, 0.1-0.6%, 0.1-0.5%, 0.1-0.4%, 0.1-0.3%, or 0.1-0.2% bwc, or any other suitable range. In certain embodiments, carbon dioxide is delivered to a mixing concrete mix at a dose of 0.01-3%, such as 0.01-1.5%, or even 0.01-1% bwc. In certain embodiments, carbon dioxide is delivered to a mixing concrete mix at a dose of 0.05-3%, such as 0.05-1.5%, or even 0.05-1% bwc. In certain embodiments, carbon dioxide is delivered to a mixing concrete mix at a dose of 0.1-3%, such as 0.1-1.5%, or even 0.1-1% bwc. It is understood that, in the case of these dosages, they refer to amount of carbon dioxide delivered to the concrete and do not include atmospheric carbon dioxide that may also be absorbed by the mix during mixing. In certain embodiments, carbon dioxide is delivered to a mix that is cement and water, with aggregates and other ingredients, if used, delivered after carbonation of the cement/water mix, or after partial carbonation of the cement/water mix. A suitable system to carbonate mixing concrete mix is present at the site, e.g., a system to convert liquid carbon dioxide to solid and gaseous carbon dioxide and deliver the solid and gaseous carbon dioxide to the mixing concrete, such as a system described in PCT Publication No. WO2020124054. Further processes and apparatus for use in carbonation of mixing concrete mixes and mixing cement mixes are described more fully in U.S. Pat. No. 10,927,042.

Alternatively or additionally, carbon dioxide from one or more carbon dioxide producers, such as carbon dioxide containing one or more contaminants as described herein, or a portion thereof, is used to carbonate a wet concrete mix, but after its initial mixing. These may be, e.g., batches that don't meet quality control at a concrete production facility, concrete produced in excess of that used at a job site that is returned to a concrete production facility, and the like. Although such wet concrete may be subject to mixing or agitation in order to maintain fluidity and postpone set and hardening, it is not considered a “mixing concrete mix” as described above, which embodiment refers to the initial mixing of the concrete ingredients. In these embodiments, carbon dioxide may be delivered to the concrete mix in any suitable form, for example, as a mixture of solid and gaseous carbon dioxide which may, e.g., be produced from liquid carbon dioxide. Any suitable dosage of carbon dioxide may be used, which is often higher than a dosage that can be used in, e.g., mixing concrete, because it is not necessary to maintain workability in the concrete e.g., returned to a facility, since it won't be poured at a job site or otherwise placed in a mold or container to which it must conform. Thus, suitable ranges of doses can be e.g., 0.001-20%, 0.001-10%, 0.001-5%, 0.001-3%, 0.001-2%, 0.001-1.5%, 0.001-1.0%, 0.001-0.8%, 0.001-0.5%, 0.001-0.1%, 0.005-20%, 0.005-10%, 0.005-5%, 0.005-3%, 0.005-2%, 0.005-1.5%, 0.005-1.0%, 0.005-0.8%, 0.005-0.5%, 0.005-0.1%, 0.01-20%, 0.01-10%, 0.01-5%, 0.01-3%, 0.01-2%, 0.01-1.5%, 0.01-1.0%, 0.01-0.8%, 0.01-0.5%, 0.01-0.1%, 0.1-20%, 0.1-10%, 0.1-5%, 0.1-3%, 0.1-2%, 0.1-1.5%, 0.1-1.4%, 0.1-1.3%, 0.1-1.2%, 0.1-1.0%, 0.1-0.9%, 0.1-0.8%, 0.1-0.7%, 0.1-0.6%, 0.1-0.5%, 0.1-0.4%, 0.1-0.3%, or 0.1-0.2% bwc, or any other suitable range. In certain embodiments, carbon dioxide is delivered to a mixing concrete mix at a dose of 0.01-6%, such as 0.01-3%, or even 0.01-2%, in certain cases 0.01-1% bwc. In certain embodiments, carbon dioxide is delivered to a mixing concrete mix at a dose of 0.05-6%, such as 0.05-3%, or even 0.05-2%, in certain cases 0.05-1% bwc. In certain embodiments, carbon dioxide is delivered to a mixing concrete mix at a dose of 0.1-6%, such as 0.1-3%, or even 0.1-2%, in certain cases 0.1-1% bwc. It is understood that, in the case of these dosages, they refer to amount of carbon dioxide delivered to the concrete and do not include atmospheric carbon dioxide that may also be absorbed by the mix during mixing. Typically, a wet mix treated in this manner is allowed to harden and may be treated to produce, e.g., aggregate for future batches of concrete. A suitable system is present to carbonate the wet concrete mix. If the site already includes a system to carbonate a mixing concrete mix, the same or similar system may be used to carbonate wet concrete mixes: typically, a different but similar system is used (e.g., a system to convert liquid carbon dioxide to gaseous and solid carbon dioxide and deliver the gaseous and solid carbon dioxide to the wet concrete mix).

Alternatively or additionally, carbon dioxide from one or more carbon dioxide producers, such as carbon dioxide containing one or more contaminants as described herein, or a portion thereof, is used to carbonate recycled concrete aggregates (RCA). Carbonation of RCA is well-known, and any suitable method may be used. In certain cases, the aggregate is carbonated in a vessel into RCA is placed, then into which gaseous carbon dioxide, such as carbon dioxide as described herein, e.g., including one or more contaminants, is introduced, generally at at least sufficient pressure to drive out most or all of air in the vessel. In certain embodiments, RCA is placed in a vessel and partially or completely covered with water (which may be carbonated wash water or clarified carbonated wash water, as described in the next section), and carbon dioxide is either present in the water already (in solution and/or in reacted form such as carbonic acid or products of carbonic acid) or is added to the water, or both, and the RCA is carbonated by the aqueous carbon dioxide and/or carbon dioxide aqueous reaction products. Dosages can include any one of those disclosed PCT Publication No. WO2020217232. It is understood that, in the case of these dosages, they refer to amount of carbon dioxide delivered to the RCA and do not include atmospheric carbon dioxide that may also be absorbed by the RCA during processing. For further description of carbonation of RCA, see PCT Publication No. WO2020217232. Carbonated RCAs can be used in any suitable manner, such as re-used in subsequent batches of concrete, for road fill layers, and the like. A suitable system is present to carbonate the RCAs, as described (e.g., air tank, aqueous tank, or any suitable system).

Alternatively or additionally, carbon dioxide from one or more carbon dioxide producers, such as carbon dioxide containing one or more contaminants as described herein, is used to carbonate concrete wash water. Concrete production operations generate wash water from various aspects of the production, transport, and use of concrete, e.g., water used at the job site after a load has been poured to wash down a drum and keep remaining concrete from setting and hardening, and/or back at a batching site to wash out a drum, and the like, that generally is not allowed to return to the environment untreated. As will be clear from context, “wash water” includes water used to clean the drum of a ready-mix truck and/or other mixers, which contains cement and aggregate, as well as such water after aggregate has been removed, e.g., in a reclaimer, but still containing solids, such as cementitious solids. Typically at least a portion of such solids are retained in the wash water for re-use in subsequent concrete batches, in some cases. Carbon dioxide may be used to treat the wash water, such as to render it suitable for disposal into water disposal systems: in certain embodiments, wash water may be carbonated and may be used as at least a portion of, or in some cases all of, mix water in a subsequent batch. In some cases there is no need to further treat wash water, beyond carbonation, for the wash water to be suitable for use as mix water in a subsequent batch. In certain embodiments, after grey (wash) water is carbonated, it is used in subsequent batches of concrete with no more than 5, 10, 15, 20, 30, 40, 50, 60, 70, 80, 90, or 95% of remaining solids removed. In certain embodiments, none of the remain solids are removed. The carbonated wash water may be combined with non-wash water, e.g., normal mix water, before or during use in a subsequent concrete batch, to provide a total amount of water used in the batch; in certain embodiments, the carbonated wash water comprises at least 5, 10, 15, 20, 30, 40, 50, 60, 70, 80, 90, 95, or 99% of the total amount of water used in the batch: in certain embodiments, 100% of the total amount of water used in the batch is carbonated wash water, excluding water used to wash down equipment and, in some cases, excluding water added at the job before or during pouring of the concrete mix. The dose of carbon dioxide to be used for wash water (either in a single truck or in a combination of more than one truck) may be expressed as an amount by weight solids, where a percentage of cement and other carbon-dioxide-reacting or -absorbing materials is known or estimated, and/or efficiency of carbonation is known or estimated, e.g., at least 1, 2, 5, 7, 10, 15, 20, 25, 30, 35, 40, 45, 50, 55, 60, 65, 70, 75, 80, 85, 90, or 95% carbon dioxide by weight solids, and/or not more than 2, 5, 7, 10, 15, 20, 25, 30, 35, 40, 45, 50, 55, 60, 65, 70, 75, 80, 85, 90, 95 or 100% carbon dioxide by weight solids. Higher doses may be used, e.g., beyond 100% by weight solids, depending on the cement content of the wash water, the expected efficiency of carbonation, etc. Re-use of the carbonated wash water avoids disposal costs and equipment, and can result in further advantages in the concrete batch in which it is used, e.g., contribute some active cement, thus requiring less new cement in the batch, and/or increased strength of the concrete compared to the same mix made without carbonated wash water (which can lead to further reduction in the amount of cement needed). Use of carbonated wash water in a mixing concrete mix can be supplemented by carbonation of the mix, as described above. Alternatively or additionally, carbonated wash water, either as is or after further suitable treatment, may be used as a source of water in which to immerse recycled concrete aggregates for carbonation (see next section), and/or as a source of additional water that may be added to a wet concrete mix before, during, or after carbonation. It is understood that, in the case of these dosages, they refer to amount of carbon dioxide delivered to the wash water and do not include atmospheric carbon dioxide that may also be absorbed by the wash water during mixing. A system is present at the site to carbonate the wash water, e.g., by providing gaseous carbon dioxide, such as a system that carbonates wash water in a recirculation loop attached to a holding tank or similar container. Wash water carbonation processes and apparatus are further described in PCT Publication No. WO2018232507.

In certain embodiments, a system and/or method is provided that includes a biogas facility and a direct air capture (DAC) facility. Methane produced at the biogas facility typically must be treated to further purify it to meet standards for introduction into a supply pipeline. A portion of this raw methane can be burned. The heat from this raw methane burn can be used 1) for electricity generation (e.g., in a steam-driven generator) and/or 2) as a heat source for regeneration of a carbon dioxide sorbent used in a DAC facility, if the sorbent is regenerated (i.e., releases captured carbon dioxide so that the sorbent is ready for reuse to capture further carbon dioxide) in a process that utilizes temperature swing, alone or in combination with other processes, as known in the art. It will be appreciated that, even if the heat generated by combustion of methane is used for electricity generation, a certain amount of waste heat will still be produced, which can be used to regenerate the sorbent. The heat may be produced in sufficient quantity to be used as is for sorbent regeneration: in certain embodiments, a heat pump may be used to elevate the temperature sufficiently to be used in sorbent regeneration, though typically the heat from the burning of raw methane can be sufficient to drive sorbent regeneration without a heat pump. In general, a DAC facility includes a system to move air (e.g., fans), a system remove water from air (e.g., dehumidifier or desiccant), and a carbon dioxide absorber system, which will typically include a sorbent, such as an amine sorbent, that reversibly binds carbon dioxide. One or more of these systems utilizes energy to power its process: e.g., the fans that move the air. Thus, methane burning at the biogas facility can be coupled with carbon dioxide capture and release at the DAC facility, with the methane burning (e.g., from raw methane) supplying one or both of electricity to power one or more parts of systems of the DAC facility, e.g., fans and/or other apparatus or process that requires electrical energy, and/or heat, e.g., waste heat, to regenerate a carbon dioxide sorbent, e.g., an amine sorbent. The carbon dioxide sorbent is then ready to absorb more carbon dioxide. The carbon dioxide from burning methane may be added to carbon dioxide already produced by the biogas facility that is being treated as described herein. The carbon dioxide released from the DAC system sorbent can also be added to that carbon dioxide.

Each of the above processes, and corresponding systems to perform the processes, may be at the same site or at different sites. For example, a concrete product site may incorporate one, two, three or all four of a system to carbonate a mixing concrete mix, a system to carbonate concrete wash water (generally, wherein at least a portion of the carbonated wash water is used as mix water in a subsequent concrete batch), a system to carbonate wet concrete mix, and/or a system to carbonate RCA. Often one, two, or all three of a first system to carbonate a mixing concrete mix, a second system to carbonate concrete wash water, and/or a third system to carbonate a wet concrete mix are at a single site. Often, a concrete product site incorporates just a system to carbonate RCAs, e.g., is dedicated to carbonating RCAs which then can be, e.g., shipped to other concrete product sites for use, or other sites for use, such as in a roadbed. In certain embodiments, a concrete product site incorporates a system to carbonate a mixing concrete mix. In certain embodiments, a concrete product site incorporates a system to carbonate a concrete wash water. In certain embodiments, a concrete product site incorporates a system to carbonate a wet concrete mix. In certain embodiments, a concrete product site incorporates a system to carbonate an RCA. In certain embodiments, a concrete product site incorporates a first system to carbonate a mixing concrete mix and a second system to carbonate a concrete wash water: in certain of these embodiments, the site may further incorporate a third system to carbonate a wet concrete mix: in certain embodiments, the site may instead further incorporate a third system to carbonate RCAs. In certain embodiments, a concrete production site incorporates a first system to carbonate a mixing concrete mix and a second system to carbonate a wet concrete mix: in certain embodiments the site may further incorporate a third system to carbonate RCAs. In certain embodiments, a concrete production site incorporates a first system to carbonate a mixing concrete mix and a second system to carbonate RCAs. A RCA carbonation site may be a separate site, or may be incorporated into a concrete production site. In certain embodiments, a concrete product site incorporates a first system to carbonate a mixing concrete mix, a second system to carbonate a concrete wash water, a third system to carbonate a wet concrete mix, and a fourth system to carbonate RCAs.

Carbon dioxide from a particular carbon dioxide producer may be used at just one concrete product site, or more than one site, such as at least 2, 3, 4, 5, 6, 7, 8, 9, 10, 12, 15, 20, or 50 concrete product sites and/or not more than 3, 4, 5, 6, 7, 8, 9, 10, 12, 15, 20, 50 or 100 concrete product sites. Each concrete product site, whether a single site or one of a plurality of sites, may use carbon dioxide from a particular carbon dioxide producer in one, two, three, or all four of carbonation of mixing concrete, carbonation of wet concrete, carbonation of concrete aggregates, and/or carbonation of wash water. Additionally or alternatively, a particular concrete product site may use carbon dioxide from just one carbon dioxide producer, or more than one carbon dioxide producer, such as at least 2, 3, 4, 5, 6, 7, 8, 9, 10, 12, 15, 20, or 50 carbon dioxide producers and/or not more than 3, 4, 5, 6, 7, 8, 9, 10, 12, 15, 20, 50 or 100 carbon dioxide producers. It will be appreciated that any suitable combination of carbon dioxide producers and concrete product sites is encompassed by embodiments herein. Thus, in certain embodiments a single carbon dioxide production site, such as a single biogas plant, a single ethanol plant, a single hydrogen production plant, or a single DAC plant or site, or other suitable carbon dioxide producer as described herein (with, in certain embodiments, a system for rendering carbon dioxide transportable at the site) provides carbon dioxide, such as carbon dioxide with one or more contaminants as described herein, to a single concrete product site, which can incorporate one, two, three, or all four of a system to carbonate mixing concrete, a system to carbonate wet concrete, a system to carbonate RCA, and/or a system to carbonate wash water. In certain embodiments, a plurality of carbon dioxide producers, each of which can be a biogas plant, an ethanol plant, a hydrogen production plant, or a DAC plant or site, or other suitable carbon dioxide producer as described herein (with, in certain embodiments, a system for rendering carbon dioxide transportable at the site) provides carbon dioxide, such as carbon dioxide with one or more contaminants as described herein, to a single concrete product site, which can incorporate one, two, three, or all four of a system to carbonate mixing concrete, a system to carbonate wet concrete, a system to carbonate RCA, and/or a system to carbonate wash water.

In embodiments in which carbon dioxide is used in one or more reactions, e.g., is sequestered, such as permanently sequestered, such as in a concrete production process or facility, the carbon dioxide may be used as is, that is, in its delivered form (after, in some cases, being processed to be suitable for transportation from its production or other facility to a sequestration facility), including any contaminants that travel with the carbon dioxide. In some cases, the carbon dioxide may be treated to remove at least a portion of one or more contaminants. In general, though, unless a contaminant or combination of contaminants interferes significantly with a carbonation process or with other processes for which the cement/concrete/concrete product is used, or is toxic and likely to be released to the environment at unacceptable levels during or after the carbonation process, it is preferred that they remain in the carbon dioxide. The fate of a contaminant in the carbon dioxide used for carbonation, in this and in other forms of carbonation, can depend on the contaminant, e.g., the chemical nature of the contaminant, and/or the method and materials of carbonation. In some cases, a contaminant may itself undergo a chemical reaction or other process to become a thermodynamically stable product. In some cases, a contaminant may not become a thermodynamically stable product: in some such cases it may be trapped, e.g., physically trapped within the concrete and unlikely to be released, at least during the lifetime of the concrete. In some cases, a contaminant may be released to the environment, e.g., atmosphere, or waste water, etc.: so long as the contaminant is not one for which such release is prohibited, or is prohibited above a certain level (and released is below this level), or for which such release is undesirable, this can be acceptable. In this case, many substances that are labeled as “contaminants” of carbon dioxide are not toxic, or not significantly toxic, but merely, e.g., render the carbon dioxide of less use or even useless for its intended use (e.g., in some industrial applications like welding), without imparting any or any significant toxicity if released to the atmosphere or otherwise released into the environment. Thus, it is possible to use carbon dioxide in this or other carbonation processes, e.g., those processes described herein, that is not considered of suitable purity for a particular use, e.g., not food grade carbon dioxide, such as carbon dioxide that is less than 99.90% pure, or, e.g., not industrial grade carbon dioxide, such as carbon dioxide used in welding which generally is considered to require 99.5% pure, to be pure enough for a carbonation process, such as a process described herein, and to not release to the environment significant quantities, e.g., a quantity that is above regulated levels, or, in some cases any, contaminants that may be of environmental and/or health concern.

In certain embodiments carbon dioxide from a biogas facilities is used and/or a biogas facility itself and/or system at one or more biogas facilities to separate carbon dioxide and/or render carbon dioxide suitable for transport is used.

### Specific Embodiments

In a first embodiment, provided herein is a method of carbon removal comprising (i) either (a) providing carbon dioxide derived from a gas comprising methane and carbon dioxide generated by anaerobic digestion of organic material at a first site, wherein at least a portion of the carbon dioxide is separated from the methane to produce a carbon dioxide portion and a methane portion, or (b) providing carbon dioxide derived from a composition generated by direct air capture of carbon dioxide at a second site by separating at least a portion of carbon dioxide from the composition; and (ii) sequestering at least part of the carbon dioxide portion at a third site by chemically reacting it or a reaction product of it to form a carbon dioxide sequestration product or products. In a second embodiment provided is the method of the first embodiment wherein the first and second sites are different from the third site. In a third embodiment provided is the method of the second embodiment further comprising, at the first site and/or the second site, processing the carbon dioxide, if necessary, to render it suitable for transport, and transporting the carbon dioxide to the third site. In a fourth embodiment, provided is the method of the third embodiment wherein the first and/or second sites are within 5, 10, 15, 20, 30, 40, 50, 60, 70, 80, 90, 100, 120, 150, 200, 250, 300, 400, 500, 700, or 1000 miles of the third site. In a fifth embodiment provided is the method of the fourth embodiment wherein the first and/or second sites are within 250 miles of the third site. In a sixth embodiment, provided is a method of any one of the first through fifth embodiments, wherein further provided is carbon dioxide derived from a gas comprising methane and carbon dioxide generated by anaerobic digestion of organic material, wherein at least a portion of the carbon dioxide is separated from the methane to produce a carbon dioxide portion and a methane portion, at one or more additional sites different from the first site, for example at least 1, 2, 3, 4, 5, 6, 7, 8, 9, or 10 sites different from the first site; and/or providing carbon dioxide derived from a composition generated by direct air capture of carbon dioxide by separating at least a portion of carbon dioxide from the composition at one or more additional sites different from the second site, for example at least 1, 2, 3, 4, 5, 6, 7, 8, 9, or 10 sites different from the second site; and/or sequestering at least part of the carbon dioxide portion provided by the first and, in some cases, additional sites similar to the first, and/or the second and, in some cases additional sites similar to the second site, by chemically reacting it or a reaction product of it to form a carbon dioxide sequestration product or products at one or more additional sites different from the third site, for example at least 1, 2, 3, 4, 5, 6, 7, 8, 9, or 10 sites different from the third site. In a seventh embodiment provided is the method of the sixth embodiment wherein one or more additional sites, similar to the third site, sequester at least part of the carbon dioxide portion from the first, second, and/or additional sites. In an eighth embodiment provided is the method of any one of the first through seventh embodiments wherein the carbon dioxide portion of (i) (a) comprises one or more contaminants. In a ninth embodiment, provided is the method of the eighth embodiment wherein the nature and/or concentration of the contaminants render it unsuitable as a food-grade carbon dioxide. In a tenth embodiment, provided is the method of the eighth or the ninth embodiment wherein the one or more contaminants comprise organic matter (e.g., fecal matter), one or more sulfur compounds (e.g., H2S, thiols), ammonia, siloxanes, or a combination thereof. In an eleventh embodiment provided is the method of any one of the eighth through tenth embodiments wherein the carbon dioxide comprises 0.01-10% contaminants (for example, % w/w). In a twelfth embodiment provided is the method of any one of the eighth through tenth embodiments wherein the impurities are present at a combined level of at least 0.001, 0.01, 0.09, 0.1, 0.2, 0.3, 0.4, 0.49, 0.5, 0.6, 0.7, 0.8, 0.9, 0.99, 1.0, 1.1, 1.2, 1.5, 1.7, 2, 2.5, 3, 3.5, 4, 4.5, 5, 6, 7, 8, 9, 10, 12, 15, or 20% (for example, % w/w), such as at least 0.09%, or in some cases at least 0.49%, such as in certain cases at least 0.99%, In a thirteenth embodiment, provided is the method of any one of the eighth through twelfth embodiments wherein at least a portion of the one or more contaminants is sequestered in the carbon dioxide sequestration product or products. In a fourteenth embodiment, provided is the method of the thirteenth embodiment wherein the one or more contaminants is permanently sequestered in the carbon dioxide sequestration product or products. In a fifteenth embodiment, provided is a method of any one of the first through fourteenth embodiments wherein the organic material comprises landfill, waste water treatment product (e.g., sewage sludge), animal manure, food waste, agricultural residue, forestry and/or forest product residue, energy crops, municipal solid waste, or a combination thereof. In a sixteenth embodiment, provided is the method of any one of the first through fifteenth embodiments wherein the carbon dioxide sequestration product or products comprise short-term sequestration products such as fuels and the like. In a seventeenth embodiment, provided is the method of any one of the first through fifteenth embodiments wherein the carbon dioxide sequestration product or products comprise permanent sequestration products. In an eighteenth embodiment, provided is the method of the seventeenth embodiment wherein the carbon dioxide sequestration product or products comprise a mineralized product. In a nineteenth embodiment provided is the method of any one of the first through fifteenth embodiments wherein the carbon dioxide sequestration product or products comprise one or more carbonated concrete production products. In a twentieth embodiment provided is the method of the nineteenth embodiment wherein the carbonated concrete production product comprises carbonated concrete, carbonated concrete wash water, carbonated recycled concrete aggregates, or a combination thereof. In a twenty-first embodiment provided is the method of any one of the first through twentieth embodiments further comprising quantifying carbon dioxide removal. In a twenty-second embodiment provided is the method of the twenty-first embodiment further comprising obtaining carbon credit based on the quantified amount of carbon dioxide removed from the atmosphere. In a twenty-third embodiment, provided is the method of any one of the first through twenty-second embodiments wherein digital information sharing of capture and utilization/storage sites. In a twenty-fourth embodiment, provided is the method of any one of the first through twenty-third embodiments, wherein at least a portion of the chemically reacting comprises formation of noncovalent bonds. In a twenty-fifth embodiment, provided is the method of any one of the first through twenty-third embodiments, wherein at least a portion of the chemically reacting comprises formation of covalent bonds.

In a twenty-sixth embodiment provided is a method comprising (i) providing carbon dioxide that contains 0.001, 0.01, 0.09, 0.1, 0.2, 0.3, 0.4, 0.49, 0.5, 0.6, 0.7, 0.8, 0.9, 0.99, 1.0, 1.1, 1.2, 1.5, 1.7, 2, 2.5, 3, 3.5, 4, 4.5, 5, 6, 7, 8, 9, 10, 12, 15, or 20% (for example, % w/w), such as at least 0.09%, or in some cases at least 0.49%, such as in certain cases at least 0.99% contaminants; and (ii) sequestering at least part of the carbon dioxide by chemically reacting it or a reaction product of it to form a carbon dioxide sequestration product or products. In a twenty-seventh embodiment provided is the method of the twenty-sixth embodiment wherein the one or more contaminants comprise one or more of organic matter (e.g., fecal matter), one or more sulfur compounds (e.g., H2S, thiols), ammonia, siloxanes, water vapor, nitrogen, oxygen, hydrogen, carbon monoxide, halogenated hydrocarbons, other volatile and semi-volatile organic compounds, heavy metals or a combination thereof. In a twenty-eighth embodiment provided is the method of the twenty-sixth or twenty-seventh embodiment wherein at least a portion of the one or more contaminants is sequestered, for example, permanently sequestered. In a twenty-ninth embodiment provided is the method of any one of the twenty-sixth through twenty-eighth embodiments wherein the one or more contaminants comprises one or more of organic matter (e.g., fecal matter), one or more sulfur compounds (e.g., H2S, thiols), ammonia, siloxanes, or a combination thereof. In a thirtieth embodiment provided is the method of any one of the twenty-sixth through twenty-ninth embodiments wherein the carbon dioxide is produced during ethanol production, biogas production, hydrogen production, direct air capture, or a combination thereof. In a thirty-first embodiment, provided is the method of any one of the twenty-sixth through thirtieth embodiments wherein the sequestering comprises contacting the carbon dioxide with concrete or a concrete product.

In a thirty-second embodiment provided is a method comprising (i) providing a gas comprising carbon dioxide wherein the carbon dioxide is derived, directly or indirectly, from atmospheric carbon dioxide; and (ii) sequestering at least part of the carbon dioxide by chemically reacting it or a reaction product of it to form a carbon dioxide sequestration product or products. In a thirty-third embodiment provided is the method of the thirty-second embodiment wherein the carbon dioxide comprises one or more contaminants. In a thirty-fourth embodiment provided is the method of the thirty-third embodiment wherein the one or more contaminants are present at a total concentration of at least 0.001, 0.01, 0.09, 0.1, 0.2, 0.3, 0.4, 0.49, 0.5, 0.6, 0.7, 0.8, 0.9, 0.99, 1.0, 1.1, 1.2, 1.5, 1.7, 2, 2.5, 3, 3.5, 4, 4.5, 5, 6, 7, 8, 9, 10, 12, 15, or 20% (for example, % w/w), such as at least 0.09%, or in some cases at least 0.49%, such as in certain cases at least 0.99% contaminants. In a thirty-fifth embodiment, provided is the method of any one of the thirty-second through thirty-thirty-fourth embodiments wherein the carbon dioxide comprises carbon dioxide obtained by DAC. In a thirty-sixth embodiment, provided is the method of any one of the thirty-second through thirty-thirty-fourth embodiments wherein the carbon dioxide comprises carbon dioxide produced by anaerobic digestion of organic material. In a thirty-seventh embodiment, provided is the method of any one of the thirty-second through thirty-thirty-fourth embodiments wherein the carbon dioxide comprises carbon dioxide produced during ethanol production, e.g., from a biomass crop, such as corn. In a thirty-eighth embodiment, provided is the method of any one of the thirty-second through thirty-thirty-fourth embodiments wherein carbon dioxide comprises carbon dioxide produced during production of H2, e.g., production of H2 from biomass. In a thirty-ninth embodiment, provided is the method of any one of the thirty-second through thirty-eighth embodiments wherein the sequestering comprises contacting the carbon dioxide with concrete or a concrete product. Further details concerning contacting the carbon dioxide with concrete or a concrete product are as provided herein.

In a fortieth embodiment provided herein is a carbon removal system comprising (i) carbon dioxide-producing facility comprising a source of carbon dioxide derived directly or indirectly from the atmosphere, wherein the facility comprises a system for processing the carbon dioxide to render it suitable for transport, if necessary; and (ii) a sequestration plant to which the carbon dioxide is transported, if necessary, and which comprises one or more systems to perform one or more processes that binds the carbon dioxide or one or more carbon dioxide reaction products with one or more reactants to produce one or more carbon dioxide sequestration products. In a forty-first embodiment provided is the system of the fortieth embodiment wherein the source is a source of biogenic carbon dioxide. In a forty-second embodiment provided is the system of the forty-first embodiment wherein the carbon dioxide-producing facility comprises a biogas plant. In a forty-third embodiment provided is the system of the forty-second embodiment wherein the biogas plant comprises a landfill gas (LFG) biogas plant, animal manure biogas plant, water resource recovery facilities (WRRF) biogas plant, food waste biogas plant, agricultural residue biogas plant, forestry and/or forest product residue biogas plant, energy crops biogas plant, municipal solid waste (MSW) biogas plant, renewable electricity biogas plant, or a combination thereof. In a forty-fourth embodiment provided is the system of any one of the fortieth through forty-third embodiments wherein the sequestration plant comprises a concrete manufacturing plant, a concrete product processing plant, or a combination thereof. In a forty-fifth embodiment provided is the system of any one of the fortieth through forty-fourth embodiments comprising a plurality of sources of atmospheric or biogenic carbon dioxide, e.g., 2, 3, 4, 5, 6, 7, 8, 9, 10, or more than 10 sources of atmospheric or biogenic carbon dioxide, a plurality of sequestration plants, e.g., 2, 3, 4, 5, 6, 7, 8, 9, 10, or more than 10 sequestration plants, or both. In a forty-sixth embodiment provided is the system of any one of the fortieth through forty-fifth embodiments wherein the source of atmospheric or biogenic carbon dioxide, or plurality thereof, and the sequestration plant, or plurality thereof, are all located within a radius of 5, 10, 15, 20, 30, 40, 50, 60, 70, 80, 90, 100, 120, 150, 200, 250, 300, 400, 500, 700, or 1000 miles. In a forty-seventh embodiment provided is the system of the forty-sixth embodiment wherein the source of atmospheric or biogenic carbon dioxide, or plurality thereof, and the sequestration plant, or plurality thereof, are within a radius of 250 miles. In a forty-eighth embodiment provided is the system of the forty-fourth embodiment, optionally comprising a plurality of sources of atmospheric or biogenic carbon dioxide and/or a plurality of concrete production or concrete product processing plants, or both, wherein the one or more systems at each of the concrete manufacturing plants or concrete product processing plants to process the carbon dioxide comprise a system to deliver at least a portion of the carbon dioxide to a mixing concrete mix, a system to deliver at least a portion of the carbon dioxide to a wet concrete mix, a system to deliver at least a portion of the carbon dioxide to concrete wash water, a system to deliver at least a portion of the carbon dioxide to recycled concrete aggregates, or a combination thereof, optionally wherein two or more, e.g., three, or all four systems are different systems. In a forty-ninth embodiment, provided herein is the system of any one of the fortieth through forty-eighth embodiments wherein the system to render the carbon dioxide transportable liquefies the carbon dioxide. In a fiftieth embodiment provided is the system of any one of the fortieth though forty-ninth embodiments wherein the system or systems to render the carbon dioxide transportable at the source and the one or more systems that performs the one or more processes are owned or controlled by a single entity. In a fifty-first embodiment provided is the system of any one of the fortieth through fiftieth embodiments further comprising components to quantify net carbon dioxide removed from the atmosphere and to generate a carbon credit.

In a fifty-second embodiment provided is a composition comprising concrete materials, carbon dioxide reaction products, and one or more contaminants, wherein the one or more contaminants was produced in a process for directly or indirectly removing carbon dioxide from the atmosphere.

In a fifty-third embodiment, provided is a method of sequestering carbon dioxide comprising (i) providing carbon dioxide produced in the production of hydrogen; and (ii) binding one or more reactants to the carbon dioxide or a carbon dioxide reaction product to produce one or more carbon dioxide sequestration products, thus sequestering the carbon dioxide.

In a fifty-fourth embodiment provided is a method of sequestering a composition comprising carbon dioxide and one or more impurities, wherein the one or more impurities comprise one or more of organic matter (e.g., fecal matter), one or more sulfur compounds (e.g., H2S, thiols), ammonia, siloxanes, water vapor, nitrogen, oxygen, hydrogen, carbon monoxide, halogenated hydrocarbons, other volatile and semi-volatile organic compounds, heavy metals or a combination thereof, in some cases one or more of organic matter (e.g., fecal matter), one or more sulfur compounds (e.g., H2S, thiols), ammonia, siloxanes, or a combination thereof, comprising reacting the composition with one or more concrete products to form one or more compositions in which the carbon dioxide and at least a portion of the one or more impurities are permanently sequestered.

In a fifty-fifth embodiment provided is a method of carbon removal comprising (i) providing carbon dioxide derived from a gas comprising carbon dioxide, wherein the carbon dioxide in the gas is generated by direct or indirect capture of carbon dioxide from the atmosphere at a first site: (ii) processing the carbon dioxide, if necessary, to render it suitable for transport: (iii) transporting the carbon dioxide to a second site; and (iv) sequestering at least part of the carbon dioxide portion at a second site by chemically reacting it or a reaction product of it to form a carbon dioxide sequestration product or products. In a fifty-sixth embodiment provided is the method of the fifty-fifth embodiment wherein the second site comprises a concrete product site and at least a portion of the carbon dioxide is sequestered by carbonation of one or more concrete products. In a fifty-seventh embodiment provided is the method of the fifty-sixth embodiment wherein chemically reacting the carbon dioxide or a reaction product of the reaction to form a carbon dioxide sequestration product or products comprises at least one of (a) carbonation of a mixing concrete mix: (b) carbonation of a wet concrete mix: (c) carbonation of concrete wash water to provide a carbonated wash water; and/or (d) carbonation of recycled concrete aggregates. In a fifty-eighth embodiment provided is the method of the fifty-sixth embodiment wherein chemically reacting the carbon dioxide or a reaction product of the reaction to form a carbon dioxide sequestration product or products comprises at least two of (a) carbonation of a mixing concrete mix: (b) carbonation of a wet concrete mix: (c) carbonation of concrete wash water to provide a carbonated wash water; and/or (d) carbonation of recycled concrete aggregates. In a fifty-ninth embodiment provided is the method of the fifty-sixth embodiment wherein chemically reacting the carbon dioxide or a reaction product of the reaction to form a carbon dioxide sequestration product or products comprises at least three of (a) carbonation of a mixing concrete mix: (b) carbonation of a wet concrete mix: (c) carbonation of concrete wash water to provide a carbonated wash water; and/or (d) carbonation of recycled concrete aggregates. In a sixtieth embodiment provided is the method of the fifty-sixth embodiment wherein chemically reacting the carbon dioxide or a reaction product of the reaction to form a carbon dioxide sequestration product or products comprises (a) carbonation of a mixing concrete mix: (b) carbonation of a wet concrete mix: (c) carbonation of concrete wash water to provide a carbonated wash water; and/or (d) carbonation of recycled concrete aggregates. In a sixty-first embodiment provided is the method of any one of the fifty-seventh through sixtieth embodiments wherein at least a portion of the carbonated wash water is used as mix water in a batch of concrete. In a sixty-second embodiment provided is the method of any one of the fifty-fifth through sixty-first embodiments wherein the first site is within 5, 10, 15, 20, 30, 40, 50, 60, 70, 80, 90, 100, 120, 150, 200, 250, 300, 400, 500, 700, or 1000 miles of the second site. In a sixty-third embodiment provided is the method of the sixty-second embodiment wherein the first site is within 250 miles of the second site. In a sixty-fourth embodiment provided is the method of any one of the fifty-fifth through sixty-third embodiments wherein one or more additional sites similar to the first site, provide carbon dioxide to the second site to be sequestered. In a sixty-sixth embodiment provided is the method of the sixty-fifth embodiment wherein one or more additional sites, similar to the second site, sequester at least part of the carbon dioxide portion from the first site. In a sixty-sixth embodiment provided is the method of any one of the fifty-fifth through sixty-fifth embodiments wherein the carbon dioxide portion of (i) comprises one or more contaminants. In a sixty-seventh embodiment provided is the method of the sixty-sixth embodiment wherein the nature and/or concentration of the contaminants render it unsuitable as a food-grade carbon dioxide. In a sixty-eighth embodiment provided is the method of the sixty-fifth or sixty-sixth embodiment wherein the one or more contaminants comprise organic matter (e.g., fecal matter), one or more sulfur compounds (e.g., H2S, thiols), ammonia, siloxanes, or a combination thereof. In a sixty-ninth embodiment provided is the method of the sixty-eighth embodiment wherein the carbon dioxide comprises 0.01-10% contaminants (e.g., w/w). In a seventieth embodiment provided is the method of any of the sixty-fifth through sixty-ninth embodiments wherein the one or more contaminants are present at a total concentration of at least 0.001, 0.01, 0.09, 0.1, 0.2, 0.3, 0.4, 0.49, 0.5, 0.6, 0.7, 0.8, 0.9, 0.99, 1.0, 1.1, 1.2, 1.5, 1.7, 2, 2.5, 3, 3.5, 4, 4.5, 5, 6, 7, 8, 9, 10, 12, 15, or 20% (for example, % w/w), such as at least 0.09%, or in some cases at least 0.49%, such as in certain cases at least 0.99% contaminants. In a seventieth embodiment provided is the method of the sixty-ninth embodiment wherein the carbon dioxide comprises at least 0.09% contaminants (e.g., w/w). In a seventy-first embodiment provided is the method of the sixty-ninth embodiment wherein the carbon dioxide comprises at least 0.99% contaminants (e.g., w/w). In a seventy-second embodiment provided is the method of the sixty-ninth embodiment wherein the carbon dioxide comprises at least 2% contaminants (w/w). In a seventy-third embodiment provided is the method of any one of the sixty-sixth through seventy-second embodiments wherein at least a portion of the one or more contaminants is sequestered in the carbon dioxide sequestration product or products. In a seventy-fourth embodiment provided is the method of the seventy-second embodiment wherein the one or more contaminants is permanently sequestered in the carbon dioxide sequestration product or products. In a seventy-fifth embodiment provided is the method of any one of the sixty-sixth through seventy-fourth embodiments wherein the one or more contaminants is permanently sequestered in the carbon dioxide sequestration product or products. In a seventy-sixth embodiment provided is the method of any one of the fifty-fifth though seventy-fourth embodiments wherein the first site comprises a biogas plant, an ethanol production plant, a direct air capture plant, a hydrogen production plant, or a combination thereof. In a seventy-seventh embodiment provided is the method of the seventy-sixth embodiment wherein the first site comprises a biogas plant. In a seventy-eighth embodiment provided is the method of the seventy-seventh embodiment wherein the biogas plant comprises wherein the biogas plant comprises a landfill gas (LFG) biogas plant, animal manure biogas plant, water resource recovery facilities (WRRF) biogas plant, food waste biogas plant, agricultural residue biogas plant, forestry and/or forest product residue biogas plant, energy crops biogas plant, municipal solid waste (MSW) biogas plant, renewable electricity biogas plant, or a combination thereof. In a seventy-ninth embodiment provided is the method of any of the fifty-fifth through seventy-eighth embodiments wherein the concrete product site comprises a concrete manufacturing plant, a concrete product processing plant, or a combination thereof.

In an eightieth embodiment provided is a method comprising (i) providing carbon dioxide derived from a gas comprising carbon dioxide and methane, wherein the gas is generated at a first biogas site: (ii) processing the carbon dioxide, if necessary, to render it suitable for transport: (iii) transporting at least a portion of the carbon dioxide to a first sequestration site; and (iv) sequestering at least part of the carbon dioxide portion at the first sequestration site by chemically reacting it or a reaction product of it to form a carbon dioxide sequestration product or products. In an eighty-first embodiment provided is the method of the eightieth embodiment wherein the second site comprises a concrete product site and at least a portion of the carbon dioxide is sequestered by carbonation of one or more concrete products. In an eighty-second embodiment provided is the method of the eighty-first embodiment wherein chemically reacting the carbon dioxide or a reaction product of the carbon dioxide to form a carbon dioxide sequestration product or products comprises at least one of (a) carbonation of a mixing concrete mix: (b) carbonation of a wet concrete mix: (c) carbonation of concrete wash water to provide a carbonated wash water; and/or (d) carbonation of recycled concrete aggregates. In an eighty-third embodiment provided is the method of the eighty-first embodiment wherein chemically reacting the carbon dioxide or a reaction product of the carbon dioxide to form a carbon dioxide sequestration product or products comprises at least two of (a) carbonation of a mixing concrete mix: (b) carbonation of a wet concrete mix: (c) carbonation of concrete wash water to provide a carbonated wash water; and/or (d) carbonation of recycled concrete aggregates. In an eighty-fourth embodiment provided is the method of the eighty-first embodiment wherein chemically reacting the carbon dioxide or a reaction product of the carbon dioxide to form a carbon dioxide sequestration product or products comprises at least three of (a) carbonation of a mixing concrete mix: (b) carbonation of a wet concrete mix: (c) carbonation of concrete wash water to provide a carbonated wash water; and/or (d) carbonation of recycled concrete aggregates. In an eighty-fifth embodiment provided is the method of the eighty-first embodiment wherein chemically reacting the carbon dioxide or a reaction product of the carbon dioxide to form a carbon dioxide sequestration product or products comprises (a) carbonation of a mixing concrete mix: (b) carbonation of a wet concrete mix: (c) carbonation of concrete wash water to provide a carbonated wash water; and (d) carbonation of recycled concrete aggregates. In an eighty-sixth embodiment provided is the method of any one of the eighty-second through eighty-fifth embodiments wherein at least a portion of the carbonated wash water is used as mix water in a batch of concrete. In an eighty-seventh embodiment provided is the method of any one of the eightieth through eighty-sixth embodiments wherein the first biogas site is within 5, 10, 15, 20, 30, 40, 50, 60, 70, 80, 90, 100, 120, 150, 200, 250, 300, 400, 500, 700, or 1000 miles of the first sequestration site. In an eighty-eighth embodiment provided is the method of the eighty-seventh embodiment wherein the first biogas site is within 250 miles of the first sequestration site. In an eighty-ninth embodiment provided is the method of any one of the eightieth through eighty-eighth embodiments further comprising (v) providing carbon dioxide derived from a gas comprising carbon dioxide and methane, wherein the gas is generated at a second biogas site, optionally also at a third, fourth, fifth, sixth, seventh, eighth, ninth, and/or tenth biogas site: (vi) processing the carbon dioxide, if necessary, to render it suitable for transport: (vii) transporting at least a portion of the carbon dioxide to the first sequestration site; and (viii) sequestering at least part of the carbon dioxide portion at the first sequestration site by chemically reacting it or a reaction product of it to form a carbon dioxide sequestration product or products. In a ninetieth embodiment provided is the method of the eighty-ninth embodiment wherein the second biogas site, optionally also the third, fourth, fifth, sixth, seventh, eighth, ninth, and/or tenth biogas site, if present, are different. In a ninety-first embodiment, provided is the method of any one of the eightieth through ninetieth embodiments further comprising transporting at least a portion of the carbon dioxide to a second sequestration site, optionally also to a third, fourth, fifth, sixth, seventh, eighth, ninth, and/or tenth sequestration site; and sequestering at least part of the carbon dioxide portion at the second sequestration site, and/or at the third, fourth, fifth, sixth, seventh, eighth, ninth, and/or tenth sequestration site, if present, by chemically reacting it or a reaction product of it to form a carbon dioxide sequestration product or products. In a ninety-second embodiment provided is the method of the ninety-first embodiment wherein the second sequestration site, and/or the third, fourth, fifth, sixth, seventh, eighth, ninth, and/or tenth sequestration site, if present, comprise concrete product sites. In a ninety-third embodiment provided is the method of the ninety-first or ninety-second embodiment wherein the second sequestration site, and/or the third, fourth, fifth, sixth, seventh, eighth, ninth, and/or tenth sequestration site, if present, are different. In a ninety-fourth embodiment provided is the method of any one of the eightieth through ninety-third embodiments wherein the carbon dioxide portion derived from a gas comprising carbon dioxide and methane comprises one or more contaminants. In a ninety-fifth embodiment provided is the method of the ninety-fifth embodiment wherein the nature and/or concentration of the contaminants render it unsuitable as a food-grade carbon dioxide. In a ninety-sixth embodiment provided is the method of the ninety-fourth or ninety-fifth embodiment wherein the one or more contaminants comprise organic matter (e.g., fecal matter), one or more sulfur compounds (e.g., H2S, thiols), ammonia, siloxanes, or a combination thereof. In a ninety-seventh embodiment provided is the method of any one of the ninety-fourth through ninety-sixth embodiments wherein the carbon dioxide comprises 0.01-10% contaminants (for example, % w/w). In a ninety-eighth embodiment provided is the method of any one of the ninety-fourth through ninety sixth embodiments wherein the one or more contaminants are present at a total concentration of at least 0.001, 0.01, 0.09, 0.1, 0.2, 0.3, 0.4, 0.49, 0.5, 0.6, 0.7, 0.8, 0.9, 0.99, 1.0, 1.1, 1.2, 1.5, 1.7, 2, 2.5, 3, 3.5, 4, 4.5, 5, 6, 7, 8, 9, 10, 12, 15, or 20% (for example, % w/w), such as at least 0.09%, In a ninety-ninth embodiment provided is the method of the ninety-eighth embodiment wherein the carbon dioxide comprises at least 0.99% contaminants (w/w). In a one hundredth embodiment provided is the method of the ninety-eighth embodiment wherein the carbon dioxide comprises at least 2% contaminants (w/w). In a one hundred and first embodiment provided is the method of any one of the sixty-sixth through seventy-second embodiments wherein at least a portion of the one or more contaminants is sequestered in the carbon dioxide sequestration product or products. In a one hundred and second embodiment, provided is the method of any one of the ninety-fourth through one hundred and first embodiments wherein the one or more contaminants is permanently sequestered in the carbon dioxide sequestration product or products. In a one hundred and third embodiment provided is the method of any one of the eightieth through one hundred second embodiments wherein the first biogas plant, and/or second, third, fourth biogas plants, if present, comprise a landfill gas (LFG) biogas plant, animal manure biogas plant, water resource recovery facilities (WRRF) biogas plant, food waste biogas plant, agricultural residue biogas plant, forestry and/or forest product residue biogas plant, energy crops biogas plant, municipal solid waste (MSW) biogas plant, renewable electricity biogas plant, or a combination thereof. In a one hundred and fourth embodiment provided is the method of any one of the ninety-second through one hundred third embodiments wherein the first concrete product site, and/or second, third, fourth concrete product site, comprises a concrete manufacturing plant, a concrete product processing plant, or a combination thereof.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of carbon removal comprising
(i) either
(a) providing carbon dioxide derived from a gas comprising methane and carbon dioxide generated by anaerobic digestion of organic material at a first site, wherein at least a portion of the carbon dioxide is separated from the methane to produce a carbon dioxide portion and a methane portion, or
(b) providing carbon dioxide derived from a composition generated by direct air capture of carbon dioxide at a second site by separating at least a portion of carbon dioxide from the composition; and
(ii) sequestering at least part of the carbon dioxide portion at a third site by chemically reacting it or a reaction product of it to form a carbon dioxide sequestration product or products.

2. The method of claim 1 wherein the first and second sites are different from the third site.

3. The method of claim 2 further comprising, at the first site and/or the second site, processing the carbon dioxide, if necessary, to render it suitable for transport, and transporting the carbon dioxide to the third site.

4. The method of claim 3 wherein the first and/or second sites are within 5, 10, 15, 20, 30, 40, 50, 60, 70, 80, 90, 100, 120, 150, 200, 250, 300, 400, 500, 700, or 1000 miles of the third site.

5. The method of claim 4 wherein the first and/or second sites are within 250 miles of the third site.

6. The method of any of claims 1-5 wherein one or more additional sites similar to the first site, and/or one or more additional sites similar to the second site, provide carbon dioxide to the third site to be sequestered.

7. The method of any of claims 1-6 wherein one or more additional sites, similar to the third site, sequester at least part of the carbon dioxide portion from the first, second, and/or additional sites.

8. The method of any previous claim wherein the carbon dioxide portion of (i) (a) comprises one or more contaminants.

9. The method of claim 8 wherein the nature and/or concentration of the contaminants render it unsuitable as a food-grade carbon dioxide.

10. The method of claim 8 wherein the one or more contaminants comprise organic matter (e.g., fecal matter), one or more sulfur compounds (e.g., H2S, thiols), ammonia, siloxanes, or a combination thereof.

11. The method of claim 10 wherein the carbon dioxide comprises 0.01-10% contaminants (w/w).

12. The method of claim 10 wherein the impurities are present at a combined level of at least 0.001, 0.01, 0.09, 0.1, 0.2, 0.3, 0.4, 0.5, 0.6, 0.7, 0.8, 0.9, 1.0, 1.1, 1.2, 1.5, 1.7, 2, 2.5, 3, 3.5, 4, 4.5, 5, 6, 7, 8, 9, 10, 12, 15, or 20%, such as at least 0.09%.

13. The method of any of claims 8-12 wherein at least a portion of the one or more contaminants is sequestered in the carbon dioxide sequestration product or products.

14. The method of claim 13 wherein the one or more contaminants is permanently sequestered in the carbon dioxide sequestration product or products.

15. The method of claim 1 wherein the organic material comprises landfill, waste water treatment product (e.g., sewage sludge), animal manure, food waste, agricultural residue, forestry and/or forest product residue, energy crops, municipal solid waste, or a combination thereof.

16. The method of any of claims 1-15 wherein the carbon dioxide sequestration product or products comprise short-term sequestration products such as fuels and the like.

17. The method of any of claims 1-15 wherein the carbon dioxide sequestration product or products comprise permanent sequestration products.

18. The method of claim 17 wherein the carbon dioxide sequestration product or products comprise a mineralized product.

19. The method of any of claims 1-15 wherein the carbon dioxide sequestration product or products comprise one or more carbonated concrete production products.

20. The method of claim 19 wherein the carbonated concrete production product comprises carbonated concrete, carbonated concrete wash water, carbonated recycled concrete aggregates, or a combination thereof.

21. The method of any previous claim further comprising quantifying carbon dioxide removal.

22. The method of 21 further comprising obtaining carbon credit based on the quantified amount of carbon dioxide removed from the atmosphere.

23. The method of any previous claim wherein digital information sharing of capture and utilization/storage sites.

24. The method of any previous claim wherein at least a portion of the chemical binding is noncovalent.

25. The method of any previous claim wherein at least a portion of the chemical binding is covalent.

26. A method comprising
(i) providing carbon dioxide that contains at least 0.09% contaminants;
(ii) sequestering at least part of the carbon dioxide by chemically reacting it or a reaction product of it to form a carbon dioxide sequestration product or products

27. The method of claim 26 wherein the one or more contaminants comprise one or more of organic matter (e.g., fecal matter), one or more sulfur compounds (e.g., H2S, thiols), ammonia, siloxanes, water vapor, nitrogen, oxygen, hydrogen, carbon monoxide, halogenated hydrocarbons, other volatile and semi-volatile organic compounds, heavy metals or a combination thereof.

28. The method of claim 26 or claim 27 wherein at least a portion of the one or more contaminants is sequestered.

29. The method of any one of claim 26-28 wherein the one or more contaminants comprises one or more of organic matter (e.g., fecal matter), one or more sulfur compounds (e.g., H2S, thiols), ammonia, siloxanes, or a combination thereof.

30. The method of any one of claim 26-29 wherein the carbon dioxide is produced during ethanol production, biogas production, hydrogen production, direct air capture, or a combination thereof.

31. The method of any one of claim 26-30 wherein the sequestering comprises contacting the carbon dioxide with concrete or a concrete product.

32. A method comprising
(i) providing a gas comprising carbon dioxide wherein the carbon dioxide is derived, directly or indirectly, from atmospheric carbon dioxide; and
(ii) sequestering at least part of the carbon dioxide by chemically reacting it or a reaction product of it to form a carbon dioxide sequestration product or products

33. The method of claim 32 wherein the carbon dioxide comprises one or more contaminants

34. The method of claim 33 wherein the one or more contaminants are present at a total concentration of at least 0.001, 0.01, 0.09, 0.1, 0.2, 0.3, 0.4, 0.5, 0.6, 0.7, 0.8, 0.9, 1.0, 1.1, 1.2, 1.5, 1.7, 2, 2.5, 3, 3.5, 4, 4.5, 5, 6, 7, 8, 9, 10, 12, 15, or 20%, such as at least 0.09%.

35. The method of claim 32 wherein the carbon dioxide comprises carbon dioxide obtained by DAC.

36. The method of claim 32 wherein the carbon dioxide comprises carbon dioxide produced by anaerobic digestion of organic material.

37. The method of claim 32 wherein the carbon dioxide comprises carbon dioxide produced during ethanol production, e.g., from a biomass crop, such as corn.

38. The method of claim 32 wherein the carbon dioxide comprises carbon dioxide produced during production of H2, e.g., production of H2 from biomass.

39. The method of claim 32 wherein the sequestering comprises contacting the carbon dioxide with concrete or a concrete product.

40. A carbon removal system comprising
(i) a carbon dioxide-producing facility comprising a source of carbon dioxide derived directly or indirectly from the atmosphere, wherein the facility comprises a system for processing the carbon dioxide to render it suitable for transport, if necessary; and
(ii) a sequestration plant to which the carbon dioxide is transported, if necessary, and which comprises one or more systems to perform one or more processes that binds the carbon dioxide or one or more carbon dioxide reaction products with one or more reactants to produce one or more carbon dioxide sequestration products.

41. The system of claim 40 wherein the source is a source of biogenic carbon dioxide.

42. The system of claim 41 wherein the carbon dioxide-producing facility comprises a biogas plant.

43. The system of claim 42 wherein the biogas plant comprises a landfill gas (LFG) biogas plant, animal manure biogas plant, water resource recovery facilities (WRRF) biogas plant, food waste biogas plant, agricultural residue biogas plant, forestry and/or forest product residue biogas plant, energy crops biogas plant, municipal solid waste (MSW) biogas plant, renewable electricity biogas plant, or a combination thereof.

44. The system of any one of claims 40-43 wherein the sequestration plant comprises a concrete manufacturing plant, a concrete product processing plant, or a combination thereof.

45. The system of any one of claims 40-45 comprising a plurality of sources of atmospheric or biogenic carbon dioxide, a plurality of sequestration plants, or both.

46. The system of any of claims 40 to 45 wherein the source of atmospheric or biogenic carbon dioxide, or plurality thereof, and the sequestration plant, or plurality thereof, are within a radius of 5, 10, 15, 20, 30, 40, 50, 60, 70, 80, 90, 100, 120, 150, 200, 250, 300, 400, 500, 700, or 1000 miles.

47. The system any one of claim 46 wherein the source of atmospheric or biogenic carbon dioxide, or plurality thereof, and the sequestration plant, or plurality thereof, are within a radius of 250 miles.

48. The system of claim 44 wherein the one or more systems at the concrete manufacturing plant or concrete product processing plant to process the carbon dioxide comprise a system to deliver at least a portion of the carbon dioxide to a mixing concrete mix, a system to deliver at least a portion of the carbon dioxide to a wet concrete mix, a system to deliver at least a portion of the carbon dioxide to concrete wash water, a system to deliver at least a portion of the carbon dioxide to recycled concrete aggregates, or a combination thereof, optionally wherein two or more, e.g., three, or all four systems are different systems.

49. The system of any one of claims 40 to 47 wherein the system to render the carbon dioxide transportable liquefies the carbon dioxide.

50. The system of any one of claims 40-48 wherein the system to render the carbon dioxide transportable at the source and the one or more systems that performs the one or more processes are owned or controlled by a single entity.

51. The system of any one of claims 40-49 further comprising components to quantify net carbon dioxide removed from the atmosphere and to generate a carbon credit.

52. A composition comprising concrete materials, carbon dioxide reaction products, and one or more contaminants, wherein the one or more contaminants was produced in a process for directly or indirectly removing carbon dioxide from the atmosphere.

53. A method of sequestering carbon dioxide comprising
(i) providing carbon dioxide produced in the production of hydrogen; and
(ii) binding the carbon dioxide with one or more reactants to the carbon dioxide or a carbon dioxide reaction product and the one or more reactants to produce one or more carbon dioxide sequestration products, thus sequestering the carbon dioxide.

54. A method of sequestering a composition comprising carbon dioxide and one or more impurities, wherein the one or more impurities comprise one or more of organic matter (e.g., fecal matter), one or more sulfur compounds (e.g., H2S, thiols), ammonia, siloxanes, water vapor, nitrogen, oxygen, hydrogen, carbon monoxide, halogenated hydrocarbons, other volatile and semi-volatile organic compounds, heavy metals or a combination thereof, in some cases one or more of organic matter (e.g., fecal matter), one or more sulfur compounds (e.g., H2S, thiols), ammonia, siloxanes, or a combination thereof, comprising reacting the composition with one or more concrete products to form one or more compositions in which the carbon dioxide and at least a portion of the one or more impurities are permanently sequestered.

55. A method of carbon removal comprising
(i) providing carbon dioxide derived from a gas comprising carbon dioxide, wherein the carbon dioxide in the gas is generated by direct or indirect capture of carbon dioxide from the atmosphere at a first site:
(ii) processing the carbon dioxide, if necessary, to render it suitable for transport:
(iii) transporting the carbon dioxide to a second site; and
(iv) sequestering at least part of the carbon dioxide portion at a second site by chemically reacting it or a reaction product of it to form a carbon dioxide sequestration product or products.

56. The method of claim 55 wherein the second site comprises a concrete product site and at least a portion of the carbon dioxide is sequestered by carbonation of one or more concrete products.

57. The method of claim 56 wherein chemically reacting the carbon dioxide or a reaction product of the reaction to form a carbon dioxide sequestration product or products comprises at least one of
(a) carbonation of a mixing concrete mix:
(b) carbonation of a wet concrete mix:
(c) carbonation of concrete wash water to provide a carbonated wash water; and/or
(d) carbonation of recycled concrete aggregates.

58. The method of claim 56 wherein chemically reacting the carbon dioxide or a reaction product of the reaction to form a carbon dioxide sequestration product or products comprises at least two of
(a) carbonation of a mixing concrete mix:
(b) carbonation of a wet concrete mix:
(c) carbonation of concrete wash water to provide a carbonated wash water; and/or
(d) carbonation of recycled concrete aggregates.

59. The method of claim 56 wherein chemically reacting the carbon dioxide or a reaction product of the reaction to form a carbon dioxide sequestration product or products comprises at least three of
(a) carbonation of a mixing concrete mix:
(b) carbonation of a wet concrete mix:
(c) carbonation of concrete wash water to provide a carbonated wash water; and/or
(d) carbonation of recycled concrete aggregates.

60. The method of claim 56 wherein chemically reacting the carbon dioxide or a reaction product of the reaction to form a carbon dioxide sequestration product or products comprises
(a) carbonation of a mixing concrete mix:
(b) carbonation of a wet concrete mix:
(c) carbonation of concrete wash water to provide a carbonated wash water; and/or
(d) carbonation of recycled concrete aggregates.

61. The method of any of claims 57 to 60 wherein at least a portion of the carbonated wash water is used as mix water in a batch of concrete.

62. The method of any of claims 55-61 wherein the first site is within 5, 10, 15, 20, 30, 40, 50, 60, 70, 80, 90, 100, 120, 150, 200, 250, 300, 400, 500, 700, or 1000 miles of the second site for example, within 200, preferably within 150, even more preferably within 100 miles.

63. The method of claim 62 wherein the first site is within 250 miles of the second site.

64. The method of any of claims 55-63 wherein one or more additional sites similar to the first site, provide carbon dioxide to the second site to be sequestered.

65. The method of claim 65 wherein one or more additional sites, similar to the second site, sequester at least part of the carbon dioxide portion from the first site.

66. The method of any of claims 55-65 wherein the carbon dioxide portion of (i) comprises one or more contaminants.

67. The method of claim 66 wherein the nature and/or concentration of the contaminants render it unsuitable as a food-grade carbon dioxide.

68. The method of claim 67 wherein the one or more contaminants comprise organic matter (e.g., fecal matter), one or more sulfur compounds (e.g., H2S, thiols), ammonia, siloxanes, or a combination thereof.

69. The method of claim 68 wherein the carbon dioxide comprises 0.01-10% contaminants (w/w).

70. The method of claim 68 wherein the carbon dioxide comprises at least 0.09% contaminants (w/w).

71. The method of claim 68 wherein the carbon dioxide comprises at least 0.99% contaminants (w/w).

72. The method of claim 68 wherein the carbon dioxide comprises at least 2% contaminants (w/w).

73. The method of any of claims 66-72 wherein at least a portion of the one or more contaminants is sequestered in the carbon dioxide sequestration product or products.

74. The method of claim 73 wherein the one or more contaminants is permanently sequestered in the carbon dioxide sequestration product or products.

75. The method of any one of claims 66 to 74 wherein the one or more contaminants is permanently sequestered in the carbon dioxide sequestration product or products.

76. The method of any of claims 55-74 wherein the first site comprises a biogas plant, an ethanol production plant, a direct air capture plant, a hydrogen production plant, or a combination thereof.

77. The method of claim 75 wherein the first site comprises a biogas plant.

78. The method of claim 77 wherein the biogas plant comprises wherein the biogas plant comprises a landfill gas (LFG) biogas plant, animal manure biogas plant, water resource recovery facilities (WRRF) biogas plant, food waste biogas plant, agricultural residue biogas plant, forestry and/or forest product residue biogas plant, energy crops biogas plant, municipal solid waste (MSW) biogas plant, renewable electricity biogas plant, or a combination thereof.

79. The method of any of claims 55 to 78 wherein the concrete product site comprises a concrete manufacturing plant, a concrete product processing plant, or a combination thereof.

80. A method comprising
(i) providing carbon dioxide derived from a gas comprising carbon dioxide and methane, wherein the gas is generated at a first biogas site:
(ii) processing the carbon dioxide, if necessary, to render it suitable for transport:
(iii) transporting at least a portion of the carbon dioxide to a first sequestration site; and
(iv) sequestering at least part of the carbon dioxide portion at the first sequestration site by chemically reacting it or a reaction product of it to form a carbon dioxide sequestration product or products.

81. The method of claim 80 wherein the second site comprises a concrete product site and at least a portion of the carbon dioxide is sequestered by carbonation of one or more concrete products.

82. The method of claim 81 wherein chemically reacting the carbon dioxide or a reaction product of the carbon dioxide to form a carbon dioxide sequestration product or products comprises at least one of
(a) carbonation of a mixing concrete mix:
(b) carbonation of a wet concrete mix:
(c) carbonation of concrete wash water to provide a carbonated wash water; and/or
(d) carbonation of recycled concrete aggregates.

83. The method of claim 81 wherein chemically reacting the carbon dioxide or a reaction product of the carbon dioxide to form a carbon dioxide sequestration product or products comprises at least two of
(a) carbonation of a mixing concrete mix:
(b) carbonation of a wet concrete mix:
(c) carbonation of concrete wash water to provide a carbonated wash water; and/or
(d) carbonation of recycled concrete aggregates.

84. The method of claim 81 wherein chemically reacting the carbon dioxide or a reaction product of the carbon dioxide to form a carbon dioxide sequestration product or products comprises at least three of
(a) carbonation of a mixing concrete mix:
(b) carbonation of a wet concrete mix:
(c) carbonation of concrete wash water to provide a carbonated wash water; and/or
(d) carbonation of recycled concrete aggregates.

85. The method of claim 81 wherein chemically reacting the carbon dioxide or a reaction product of the carbon dioxide to form a carbon dioxide sequestration product or products comprises
(a) carbonation of a mixing concrete mix:
(b) carbonation of a wet concrete mix:
(c) carbonation of concrete wash water to provide a carbonated wash water; and
(d) carbonation of recycled concrete aggregates.

86. The method of any of claims 82 to 85 wherein at least a portion of the carbonated wash water is used as mix water in a batch of concrete.

87. The method of any of claims 80 to 86 wherein the first biogas site is within 5, 10, 15, 20, 30, 40, 50, 60, 70, 80, 90, 100, 120, 150, 200, 250, 300, 400, 500, 700, 1000 miles of the first sequestration site.

88. The method of claim 87 wherein the first biogas site is within 250 miles of the first sequestration site.

89. The method of any of claims 80 to 88 further comprising
(v) providing carbon dioxide derived from a gas comprising carbon dioxide and methane, wherein the gas is generated at a second biogas site, optionally also at a third biogas site, and further optionally also at a fourth biogas site:
(vi) processing the carbon dioxide, if necessary, to render it suitable for transport:
(vii) transporting at least a portion of the carbon dioxide to the first sequestration site; and
(viii) sequestering at least part of the carbon dioxide portion at the first sequestration site by chemically reacting it or a reaction product of it to form a carbon dioxide sequestration product or products.

90. The method of claim 89 wherein the second biogas site, the third biogas site, if present, and the fourth biogas site, if present, are different.

91. The method of any of claims 80 to 90 further comprising transporting at least a portion of the carbon dioxide to a second sequestration site, optionally also to a third sequestration site, optionally also to a fourth sequestration site; and sequestering at least part of the carbon dioxide portion at the second sequestration site, and/or at the third sequestration site if present, and/or at the fourth sequestration site if present, by chemically reacting it or a reaction product of it to form a carbon dioxide sequestration product or products.

92. The method of claim 91 wherein the second sequestration site, the third sequestration site, if present, and the fourth sequestration site, if present, comprise concrete product sites.

93. The method of claim 91 or claim 92 wherein the second sequestration site, the third sequestration site, if present, and the fourth sequestration site, if present, are different.

94. The method of any of claims 80-93 wherein the carbon dioxide portion derived from a gas comprising carbon dioxide and methane comprises one or more contaminants.

95. The method of claim 94 wherein the nature and/or concentration of the contaminants render it unsuitable as a food-grade carbon dioxide.

96. The method of claim 94 or claim 95 wherein the one or more contaminants comprise organic matter (e.g., fecal matter), one or more sulfur compounds (e.g., H2S, thiols), ammonia, siloxanes, or a combination thereof.

97. The method of any one of claims 94 to 96 wherein the carbon dioxide comprises 0.01-10% contaminants (w/w).

98. The method of any one of claims 94 to 96 wherein the carbon dioxide comprises at least 0.09% contaminants (w/w).

99. The method of claim 98 wherein the carbon dioxide comprises at least 0.99% contaminants (w/w).

100. The method of claim 98 wherein the carbon dioxide comprises at least 2% contaminants (w/w).

101. The method of any of claims 66-72 wherein at least a portion of the one or more contaminants is sequestered in the carbon dioxide sequestration product or products.

102. The method of any of claims 94 to 101 wherein the one or more contaminants is permanently sequestered in the carbon dioxide sequestration product or products.

103. The method of any of claims 80 to 102 wherein the first biogas plant, and/or second, third, fourth biogas plants, if present, comprise a landfill gas (LFG) biogas plant, animal manure biogas plant, water resource recovery facilities (WRRF) biogas plant, food waste biogas plant, agricultural residue biogas plant, forestry and/or forest product residue biogas plant, energy crops biogas plant, municipal solid waste (MSW) biogas plant, renewable electricity biogas plant, or a combination thereof.

104. The method of any of claims 92 to 103 wherein the first concrete product site, and/or second, third, fourth concrete product site, comprises a concrete manufacturing plant, a concrete product processing plant, or a combination thereof.
